(19) United States
(12) Patent Application Publication
(10) Pub. No.: US 2024/0216547 A1
(43) Pub. Date: Jul. 4, 2024

(54) USE OF FLUORESCEIN DICARBONATE DERIVATIVES AS A CELL MARKER

(71) Applicants: UNIVERSITE JEAN MONNET SAINT ETIENNE, SAINT ETIENNE (FR); CENTRE NATIONAL DE LA RECHERCHE SCIENTIFIQUE, PARIS (FR); UNIVERSITE DE STRASBOURG, STRASBOURG (FR); CENTRE HOSPITALIER UNIVERSITAIRE, SAINT ETIENNE (FR)

(72) Inventors: Philippe GAIN, LYON (FR); Gilles THURET, SAINT BONNET LES OULES (FR); Emilie COURRIER, MORNANT (FR); Zhiguo HE, SAINT-ETIENNE (FR); Gilles ULRICH, SOUFFELWEYERSHEIM (FR); Antoinette DE NICOLA, STRASBOURG (FR); Corantin MAURIN, SAINT-ETIENNE (FR); Corentin MARET, STRASBOURG (FR)

(21) Appl. No.: 18/558,054

(22) PCT Filed: Apr. 28, 2022
(86) PCT No.: PCT/EP2022/061415

§ 371 Date: Oct. 30, 2023

(30) Foreign Application Priority Data
Apr. 28, 2021 (FR) FR2104430

(51) Int. Cl. A61K 49/00; G01N 21/64

(52) U.S. Cl. CPC A61K 49/0043; A61K 49/0073; G01N 21/6428; G01N 2021/6439

(57) ABSTRACT

The invention relates to fluorescein derivative compounds and the use thereof as fluorescent markers. Said markers can be used in particular in biology and in medicine, particularly in the field of ophthalmology. The non-fluorescent fluorescein derivatives are activated in the cell, thereby allowing the detection of living cells as well as the quantification of cell viability by fluorescence, without being toxic to cells.

### FIELD OF THE INVENTION

The present invention relates to fluorescein derivative compounds, and use thereof as fluorescent markers. Said markers can be used in particular in biology and in medicine, particularly in the field of ophthalmology. The non-fluorescent fluorescein derivatives are activated in the living cell, thereby allowing the detection of living cells as well as the quantification of cell viability by fluorescence, without being toxic to cells.

### BACKGROUND OF THE INVENTION

Corneal transplantation is the most common transplant in the world among all cell and organ transplants with more than 100000 transplants performed each year. The cornea, the only transparent tissue in the body, plays an essential role in the refraction of light which is directed towards the retina. An alteration of this cornea leads to a reduction in visual acuity, or even loss of vision. Corneal transplantation is therefore necessary in many pathologies such as Fuchs dystrophy and bullous dystrophies, infectious keratitis and keratoconus, but is also used in cases of perforating trauma such as accidents or burns.

Corneas for transplantation are collected from donors and stored in eye banks, also called cornea banks. Before carrying out the transplant, it is essential to check the quality of the graft, several factors may be responsible for a possible alteration such as the state of health of the donor, the harvesting conditions and/or the storage conditions. To this end, it is essential that the viability of endothelial cells in corneal grafts be measured.

Many techniques for quantifying cell viability exist, but these tests are generally destructive, toxic or imperfect, that is to say they do not allow a precise, high-quality estimation of the viability of the sample.

Among the methods for measuring cell viability, mention may be made of the use of stains such as Trypan blue (CAS [75-57-1]) and tetrazolium salts (CAS [298-93-1]), or fluorophores such as acridine orange (CAS [260-94-6]) and DAPI or Di Amido Phenyl Indol (CAS [28718-90-3]). Still other compounds allow both staining and visualization of cells by fluorescence. This is the case for the compound resazurin (CAS [550-82-3]).

However, these stains or fluorophores do not allow measurement of cell viability without altering the cells and therefore the analyzed samples.

Indeed, the stains diffuse throughout the cells. Living cells exclude them while non-viable cells absorb them and become visible. A first limitation to their use is therefore the overestimation of the proportion of living cells since only cells at an advanced stage of cell death are detected. A second limitation is the intensity of the staining which is often weak making the cells barely, if ever, visible (Graefe's Archives for Clinical and Experimental Ophthalmology 1986, 224, 428-434).

Fluorophores allow to distinguish living cells, but their accumulations within cells ultimately lead to cell death.

Another approach to measuring cell viability is the use of fluorescent probes comprising one or more fluorochrome groups which are released following a cleavage reaction. The cells then become visible by fluorescence and can be easily studied under fluorescence microscopy (Anal. Chem., 2019, 91, 2255-2259; Biochemistry: Rottman and Papermaster, 1966, 55, 134-141). Such compounds are also used in other technical fields (JP 2009-006635, JP 2005-091802).

The use of these fluorescent probes has many advantages. It is a method which is selective, quick and easy to implement. In addition, fluorescent probes allow to work on living cells in vitro or in vivo. The signal emitted is generally of good quality: it is specific, durable and strong. One example of a widely used fluorescent probe for measuring cell viability is Calcein-AM.

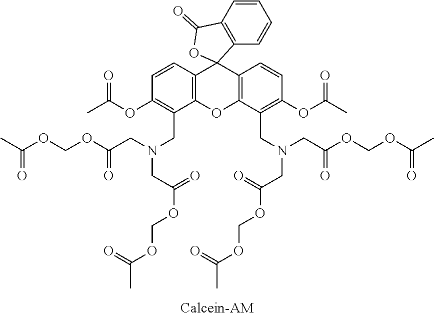 [SMILES: CC(=O)OCOC(=O)CN(CC(=O)OCOC(C)=O)Cc1c(OC(C)=O)ccc2c1Oc1c(ccc(OC(C)=O)c1CN(CC(=O)OCOC(C)=O)CC(=O)OCOC(C)=O)C21OC(=O)c2ccccc21]

This molecule enters cells, is hydrolyzed by esterases and the fluorochrome is released. However, alongside the release of the fluorochrome, formaldehyde molecules are produced, causing toxicity for the cell. The use of calcein-AM is therefore limited, if not impossible, for applications where the preservation of the viability of the studied cells or tissues is sought (IOVS, 2011, 52, 6018-6025).

There is therefore a need to develop new fluorescent markers for the quantification of cell viability while preserving this viability.

The inventors have thus developed a new marker, called in the present invention “fluorescein dicarbonate”. This fluorescent probe allows objective, precise, and non-toxic measurement of cell viability.

### BRIEF DESCRIPTION OF THE INVENTION

The invention relates to a compound of general formula (I),

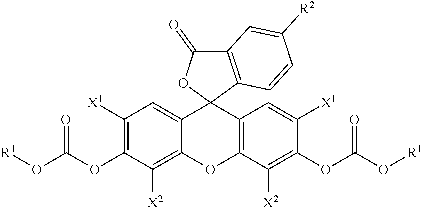

characterized in that:
R¹ is selected from
   a C₃-C₂₀ alkyl,
   a polyethylene glycol or a polypropylene glycol,
   malic acid derivatives and corresponding esters,
   sugars, and
   natural polysaccharides;
X¹ is selected from H, Cl, F, Br and NO₂;
X² is selected from H, Cl, Br and N(CH₂CO₂H)₂;
R² is selected from H, NCS and CO₂R³;
R³ is selected from H or a C₁-C₃ alkyl;

and its pharmaceutically acceptable salts.

The invention also covers the use of the compound of general formula (I) as a cell marker, and preferably as a cell viability marker.

The invention further relates to a pharmaceutical composition comprising the compound of general formula (I) and a pharmaceutically acceptable excipient and/or carrier.

The invention finds its application in the field of human, animal and plant biology. It is preferentially used in the field of ophthalmology.

Finally, a process for preparing the compound of general formula (I) is described, the process comprising the steps of: a) reaction between a diphenol of formula (II)

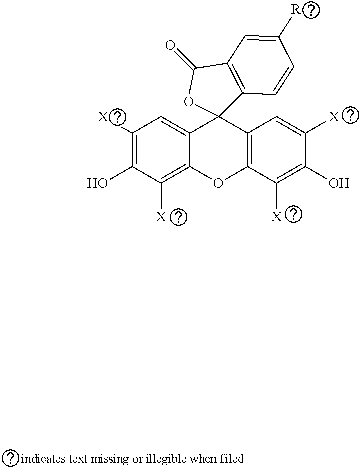

with phosgene or a derivative thereof in the presence of base, and b) reaction of the obtained intermediate (III).

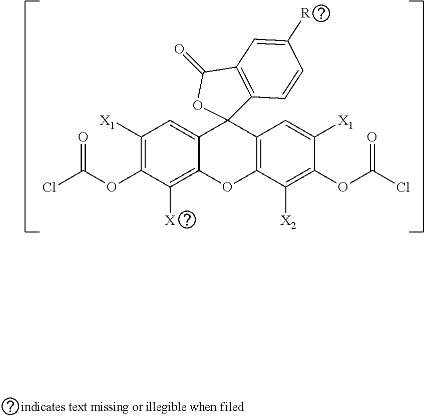

with a primary or secondary alcohol of formula (IV) R¹OH, in the presence of base, with the groups R¹, R², X¹, X² as defined previously.

### DETAILED DESCRIPTION OF THE INVENTION

The invention relates to a compound of general formula (I),

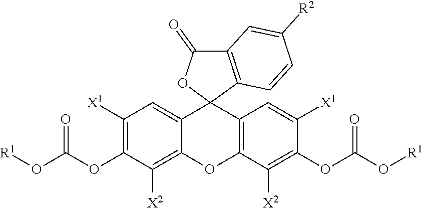

characterized in that:
R¹ is selected from
   a C₃-C₂₀ alkyl,
   a polyethylene glycol, a polypropylene glycol,
   malic acid derivatives and corresponding esters,
   sugars, and
   natural polysaccharides;
X¹ is selected from H, Cl, F, Br and NO₂;
X² is selected from H, Cl, Br and N(CH₂CO₂H)₂;
R² is selected from H, NCS and CO₂R³;
R³ is selected from H or a C₁-C₃ alkyl;

and its pharmaceutically acceptable salts.

These compounds, also called “fluorescein dicarbonate” in the context of the invention, consist of a fluorochrome, fluorescein (CAS [2321-07-5]) or a corresponding derivative, and carbonate groups of the type R¹—O—C(═O)—O which give molecules their properties of solubility and stability.

### Definitions

The compounds of general formula (I) or pharmaceutically acceptable salts may include one or more stereocenters, each of which may exist independently of the others in an R or S configuration. The compounds described are therefore in racemic or optically active forms.

The term “pharmaceutically acceptable salts” designates derivatives of compounds of general formula (I) for which the acid and/or base groups exist in their salt form. Pharmaceutically acceptable salts in the context of the present invention comprise conventional non-toxic salts. The pharmaceutically acceptable salts according to the invention will appear obvious to the person skilled in the art. Examples of salts that can be used are calcium chloride, potassium chloride, magnesium chloride, hydrogen phosphate, phosphate, edetate, citrate, lactate, hyaluronate, sodium borate, and stearate.

The term “alkyl” designates a linear and/or branched carbon chain. The term “Cₐ-C_{b}”, where a and b are integers, corresponds to the number of carbon atoms. Thus the term “C₃-C₂₀alkyl” designates a linear or branched chain comprising 3 to 20 carbon atoms. Examples of C₃-C₂₀ alkyl groups, but without limitation, are propyl, iso-propyl, butyl, iso-butyl, sec-butyl, tert-butyl, pentyl, iso-pentyl, hexyl, iso-hexyl, heotyl, octyl, nonyl, decyl, undecyl and dodecyl.

The term “polyethylene glycol” designates, in the context of the present invention, polyethylene glycols or poly(ethylene oxide)s with an average molar mass ranging from 200 to 8000 g/mol. As for the term “polypropylene glycol”, this name groups together, in the context of the present invention, polymer compounds of the propylene glycol type having an average molar mass ranging from 200 to 600 mol/g.

“Malic acid derivatives and corresponding esters” means in the context of the present invention a group defined by the general formula (V),

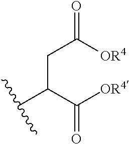

for which R⁴ and R^{4′} are independently selected from hydrogen or a C₁-C₆ alkyl.

The term “sugars” refers to the compounds glucose, fructose, galactose, mannose, sucrose, lactose, maltose and sorbitol.

“Natural polysaccharides” means in the context of the present invention polysaccharides of plant origin, such as cellulose, starch, agarose, alginate and inulin, and polysaccharides of animal origin such as hyaluronic acid or hyaluronate, chitin and chitosan.

Fluorescein can exist in two tautomeric forms: a closed form and an open form (formulas VI and VII).

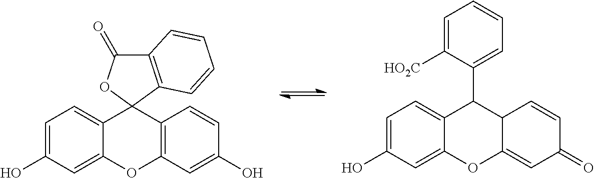 [SMILES: O=C1OC2(c3ccc(O)cc3Oc3cc(O)ccc32)c2ccccc21 ; O=C1C=CC2C(=C1)Oc1cc(O)ccc1C2c1ccccc1C(=O)O]

“Corresponding fluorescein derivative” means in the context of the present invention the fluorochrome group of fluorescein substituted by the groups X¹, X² and R² as defined previously and represented by the structure below (formula VIII). As previously described for fluorescein, it is understood that the corresponding fluorescein derivatives can exist in their tautomeric form.

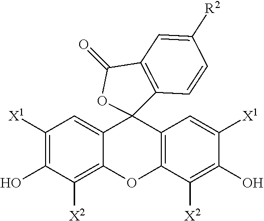

### Compounds

The present invention relates to a compound of general formula (I),

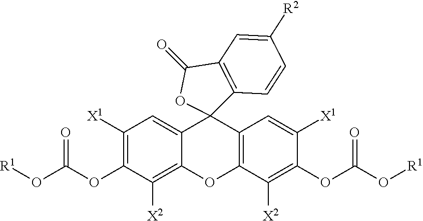

characterized in that:
R¹ is selected from
   a C₃-C₂₀ alkyl,
   a polyethylene glycol or a polypropylene glycol,
   malic acid derivatives and corresponding esters,
   sugars, and
   natural polysaccharides;
X¹ is selected from H, Cl, F, Br and NO₂;
X² is selected from H, Cl, Br and N(CH₂CO₂H)₂;
R² is selected from H, NCS and CO₂R³; and
R³ is selected from H or a C₁-C₃ alkyl;

and its pharmaceutically acceptable salts.

In one embodiment, the group R¹ is a C₅-C₁₀ alkyl. In a preferred embodiment, R¹ is selected from the n-pentyl and n-decyl groups. The alkyl groups can be linear or branched, thus in a particular embodiment, the group R¹ is a linear C₃-C₂₀ alkyl.

In another embodiment, R¹ is a derivative of malic acid or a corresponding ester, that is to say that R¹ is defined by formula (V)

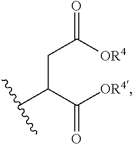

in which R⁴ and R^{4′} are selected from hydrogen or C₁-C₆ alkyl. The groups R⁴ and R^{4′} may be different or identical. In one embodiment, the groups R⁴ and R^{4′} are identical. In a preferred embodiment, R⁴ and R^{4′} are hydrogens. In another preferred embodiment, R⁴ and R^{4′} are a C₁-C₆ alkyl, preferably R⁴ and R^{4′} are ethyl.

In one embodiment, the group R² is hydrogen.

In one embodiment, the group X¹ is hydrogen.

In one embodiment, the group X² is hydrogen.

In one embodiment, the groups R², X¹ and X² are hydrogens.

### Uses

The compounds of general formula (I) or pharmaceutically acceptable salts are used for cell marking, as fluorescent markers, and more particularly for the marking of cell viability.

The compounds of the present invention comprise within their structure a fluorochrome: fluorescein or a corresponding derivative. The hydrolysis of the carbonate groups R¹—O—C(═O)—O by esterases after penetration into the cell, releases the fluorochrome as well as by-products in particular carbon dioxide and the R₁—OH type alcohol.

Groups R¹ as defined for the compounds of formula (I) or the pharmaceutically acceptable salts are preferably groups that are not toxic to cells.

The fluorescence emitted by the fluorochrome thus released is visible by techniques well known to the person skilled in the art. Visualization of fluorescence can be done with the naked eye or using devices such as glasses or microscopes, and generally requires the application of light to the fluorophore at a wavelength generally comprised between 480 and 520 nm.

The hydrolysis of the compounds of the present invention takes place at the cytoplasm. In the absence of esterase reactivity, no cleavage reaction occurs and therefore no fluorescence or staining of the cells is observed. The compounds of general formula (I) or the pharmaceutically acceptable salts are thus used for the quantification of cell viability. Indeed, the activity of ubiquitous esterases being proportional to the viability of the cells, the intensity of the signal emitted by the fluorochrome released is also proportional to the cellular activity and only living cells are detected.

The absence of cellular toxicity allows use of the compounds of the present invention both in vitro and in vivo without altering the cells, and while allowing their viability to be preserved. The products released during the hydrolysis of the carbonate groups R¹—O—C(═O)—O are carbon dioxide, alcohols of type R¹—OH as well as the fluorochrome: fluorescein or a corresponding derivative.

The invention also relates to a method of cellular marking of a set of cells which comprises the steps of contacting the cells with a compound of general formula (I) according to the invention, then of assessing the presence of stain or fluorescence in the set of cells.

The invention also relates to a method for assessing the cellular viability of a set of cells which comprises the steps of contacting the cells with a compound of general formula (I) according to the invention, then assessing the presence of staining or fluorescence of the cells. The fluorescence or staining which is the result of cell lysis of the compound of general formula (I) releasing the chromophore or fluorophore derived from fluorescein allows to conclude as to the presence of living cells.

In a particular mode of use of the compounds of general formula (I) according to the invention, the implementation of the method according to the invention comprises the identification and counting of the number of living cells in the set of cells.

The means for identifying cells and counting them are well known to the person skilled in the art. The cells, previously marked using the compounds of the present invention, are visualized using a fluorescence microscope or macroscope or with any other apparatus allowing to detect fluorescence. A light source excites the fluorophore at a wavelength comprised between 480 and 520 nm, preferably between 490 and 510 nm, more preferably between 490 and 500 nm. Advantageously, the light source excites the fluorophore at a wavelength of 495 nm. After excitation, the fluorophore releases energy in the form of fluorescent light, the wavelength of which is comprised between 500 and 540 nm, preferably between 510 and 530 nm, even more preferably at 520 nm. Marked cells become visible and can be counted.

The set of cells can be a sample taken from a living organism, in particular a biological tissue, or even an organ or part of an organ in the same living organism. Mention will be made of examples of grafts and in particular corneal grafts, bone marrow grafts and kidney grafts.

The living organisms are advantageously animals, in particular mammals, and more particularly man.

The use or method according to the invention can be implemented with the compound of general formula (I) as the sole marker of cell viability. Alternatively, it can be used with other known or future markers, in particular markers which allow to identify dead cells, so as to increase the contrast and facilitate the identification and counting of viable cells. Among these other known markers, mention can for example be made of Trypan blue.

The compounds of the present invention used as a cellular marker and preferably as a marker of cell viability find applications in numerous fields such as scientific research, cell and tissue therapy units, or else in the medical field with use for clinical diagnoses. The absence of cellular toxicity of the compounds of the present invention thus allows quantification of cell viability without altering the analyzed tissues and/or cells. They can therefore be used for in vitro, ex vivo or else in vivo studies.

In veterinary or human medicine, the compounds can be used in particular to measure the viability of a cellular and/or tissue graft before its implantation, or to monitor the evolution of a pathological process, for example in the field of oncology.

In the context of the present invention, the cells targeted by the marking are in particular endothelial cells, and more particularly corneal endothelial cells. In one embodiment, the cells of interest are hematopoietic cells.

In the field of ophthalmology, the compounds of the present invention find particular application in measuring the endothelial cell viability of corneal grafts intended for corneal transplantation, by staining corneal endothelial cells and enumerating living cells.

### Compositions and Pharmaceutical Compositions

Although it is possible for the active ingredient to be used alone, it can also be used in the form of a composition, in particular a pharmaceutical composition.

The compositions according to the invention comprise a compound of general formula (I) according to the invention and a carrier suitable for the use which will be made of the compound. For the preferred uses and methods of in vitro or in vivo cell marking, it is understood that the appropriate carrier must not affect the viability of the cells. It is understood that the compositions according to the invention do not comprise toxic substances, or at least in toxic doses, which would affect the viability of the cells. The person skilled in the art is familiar with the different carriers and excipients that can be used to allow the compound of general formula (I) to be applied to cells, and will know how to select them to promote the implementation of methods for verifying cell viability. A preferred carrier is water.

As used in the context of the present invention, the term “pharmaceutical composition” designates a mixture of at least one active compound with at least one pharmaceutically acceptable excipient and/or carrier. These pharmaceutical compositions are suitable for in vitro, ex vivo and in vivo use of the compounds of general formula (I) according to the invention.

Thus, the pharmaceutical compositions of the invention comprise a compound of general formula (I) or a pharmaceutically acceptable salt as active substance and a pharmaceutically acceptable excipient and/or carrier.

The excipient and the carrier are “pharmaceutically acceptable” in the sense that they are compatible with the other ingredients of the composition and are non-toxic. Their use allows in particular to facilitate the preparation, storage and administration of the active compound. Such excipients and carriers are well known to the person skilled in the art, described in particular in the French or European pharmacopoeia.

Pharmaceutically acceptable excipients and carriers comprise all solvents, dispersion media, coatings, antibacterial and antifungal agents, isotonic agents, absorption agents, and others that are physiologically compatible.

Examples of pharmaceutically acceptable excipients and carriers are water, saline solutions, alcohols (glycerol, glycols), polyethers (polyethylene glycols, propylene glycols, poly(oxy)ethylene glycols and their derivatives), polyethoxylated derivatives of castor oil (Kolliphor® EL, Cremophor® EL, and derivatives), hyaluronic acid, sodium hyaluronate, chondroitin sulfate, and in particular sodium chondroitin sulfate, carbomers, and in particular carbomer 974P, poloxamers, poloxamines, dextrans, vegetable oils (soybean oil, rapeseed oil, sunflower oil, olive oil, sweet almond oil, cottonseed oil, castor oil) and mineral oils (vaseline, paraffin), silicones, gelatins, agaroses (agar-agar), alginates, pectins, tragacanth, karaya gum, xanthan gum, carrageenan, carbohydrates (sucrose, lactose, amylose), starches (corn starch, rice starch, potato starch, wheat starch), fatty acid esters, cellulose and its derivatives (methylcellulose, carboxymethyl cellulose, hydroxymethylcellulose, hydroxyethylcellulose, hydroxypropylcellulose), etc. In a preferred embodiment, the pharmaceutically acceptable excipient and/or carrier is selected from water, polyethoxylated derivatives of castor oil, hyaluronic acid, sodium hyaluronate, sodium chondroitin sulfate, and carbomers.

The pharmaceutical compositions can be prepared by any method known to the person skilled in the art. An appropriate amount of the compound of general formula (I) is mixed with a pharmaceutically acceptable excipient and/or carrier to obtain the desired formulation which must be compatible with the mode of administration.

The composition according to the invention is advantageously a solution comprising the compound of general formula (I), in particular a solution, a suspension, an emulsion, a gel or an ointment or else a film. Preferably, the composition according to the invention is an aqueous composition. In one embodiment, the pharmaceutical composition is in the form of a solution, a gel or a film.

In one embodiment, the pharmaceutical composition is formulated for topical administration, and preferably for ophthalmic administration. The pharmaceutical composition can thus be formulated in the form of eye drops, collyria or eye instillations, creams, ointments, gels and hydrogels.

Liquid compositions, which are pharmaceutical or not, can also be prepared extemporaneously by mixing the compound of formula (I) according to the invention, alone or in composition form, with the liquid carrier appropriate for its use. In this case, the composition which comprises the compound of general formula (I) can be in solid form (powder or tablets in particular) or in the form of a concentrated liquid.

The pharmaceutical composition of the present invention is preferably in the form of an aqueous composition. It is particularly suitable for use in ophthalmology.

The pharmaceutical or non-pharmaceutical composition thus has a physiologically compatible pH, that is to say a pH comprised between 5 and 8. Therefore, generally it comprises a buffer suitable for ophthalmic use, known to the person skilled in the art.

The compositions of the invention may include other usual adjuvants used for the preparation of such pharmaceutical compositions such as co-solvents, softeners, antioxidants, opacifiers, stabilizers, ionic or non-ionic thickeners, surfactants, viscosity agents, osmoprotective agents, penetration agents, gelling agents, silicones, anti-foaming agents, moisturizing agents, vitamins, perfumes, preservatives, fillers, sequestrants, stains, bases or acids necessary for pH regulation, or any other ingredient usually used for the preparation of ophthalmic compositions.

The preservative agents generally used in topical compositions (cosmetics, pharmaceuticals, ophthalmics, etc.) to avoid their contamination by germs, are well known to the person skilled in the art, such as quaternary ammoniums, in particular benzalkonium chloride, alkyl-dimethyl-benzylammonium, cetrimide, cetylpyridinium chloride, benzododecinium bromide, benzothonium chloride, cetalkonium chloride, mercurial preservatives, such as phenylmercuric nitrate/acetate/borate, thiomersal, alcohol preservatives, such as chlorobutanol, benzyl alcohol, phenylethanol, phenylethyl alcohol, carboxylic acids, such as sorbic acid, phenols, in particular methyl/propyl paraben, amidines, for example chlorhexidine digluconate and/or chelating agents such as EDTA alone or in combination with at least one other preservative.

In the absence of preservatives, the composition must undergo particular treatment during its preparation and packaging so as to avoid and prevent contamination by pathogens. These treatments and procedures are well known to the person skilled in the art. In this sense, a composition without preservatives according to the invention is distinguished from a simple composition comprising the same ingredients and obtained without showing particular precautions or describing steps of the process allowing to obtain this sterility feature of pharmaceutical compositions according to the invention, in particular ophthalmic compositions.

The compounds of the present invention may be used in combination with one or more therapeutic agents. The administration of different active substances can be simultaneous, sequential or spaced overtime.

### Preparation Process

The synthesis of the compounds of general formula (I) is carried out in two successive steps from diphenol (II),

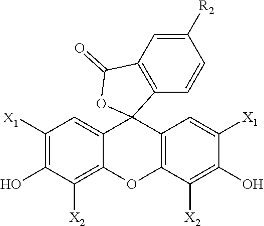

without isolating the intermediate compound (III)

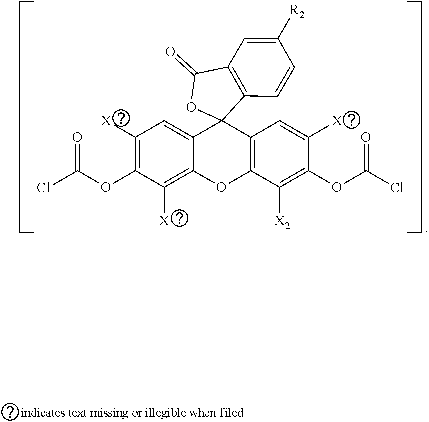

The first step is the reaction of diphenol (II) with diphosgene (trichloromethyl chloroformate, CAS [503-38-8]), or a derivative such as phosgene (carbonyl dichloride, CAS [75-44-5]) or triphosgene (bis(trichloromethyl) carbonate, CAS [32315-10-9]). In a preferred embodiment, the diphenol reacts with diphosgene.

The reaction temperature is comprised between 40 and 80° C. The reaction temperature is preferably comprised between 50 and 70° C., more preferably between 55 and 65° C. In one embodiment, the reaction temperature is comprised between 57 and 63° C., and more preferably the reaction temperature is approximately 60° C.

The reaction is carried out in an anhydrous organic solvent, and preferably in a chlorinated solvent, such as chloromethane, dichloromethane, chloroform, tetrachloromethane, trichloroethylene, 1,1,1-trichloroethane, 1,2-dichloroethane, etc. In a preferred embodiment, the reaction solvent is 1,2-dichloroethane. Other solvents in anhydrous form such as diethyl ether, dibutyl ether, tetrahydrofuran (THF) and dioxane can also be used.

A base can optionally be used for this reaction. Bases of the hindered tertiary and/or secondary amine type (non-nucleophilic) will be selected, such as triethylamine, isopropylamine, diisopropylamine, N,N-diisopropylethylamine, pyrrolidine, pyridine, etc. In one embodiment, the use of triethylamine or diisopropylethylamine will be preferred.

The intermediate compound (III),

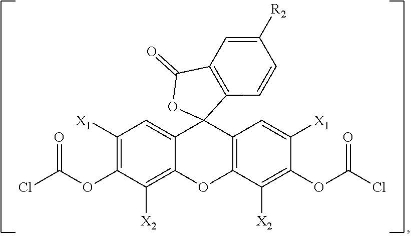

is thus obtained after evaporation of the reaction medium.

In a second step, the latter reacts with an alcohol of general formula (IV), R¹OH, to form the compound of general formula (I),

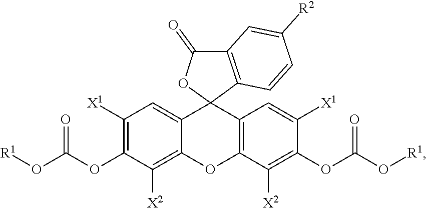

or the pharmaceutically acceptable salts.

The condensation reaction takes place at a temperature comprised between 50 and 90° C., preferably between 60 and 80° C. In one embodiment, the reaction temperature is comprised between 65 and 75° C., preferably between 68 and 72° C. In a preferred embodiment, a temperature of approximately 70° C. will be chosen.

The reaction solvent is an anhydrous organic solvent, preferably a chlorinated solvent, such as chloromethane, dichloromethane, chloroform, tetrachloromethane, trichloroethylene, 1,1,1-trichloroethane, 1,2-dichloroethane, etc. In a preferred embodiment, the reaction solvent is 1,2-dichloroethane. Other solvents in anhydrous form such as diethyl ether, dibutyl ether, THF and dioxane can also be used.

The reaction takes place in the presence of base. A tertiary amine type base will preferably be used, such as triethylamine, isopropylamine, diisopropylamine, N,N-diisopropylethylamine, pyrrolidine, pyridine, etc. In one embodiment, triethylamine or diisopropylethylamine will be the bases selected for the reaction.

The products of general formula (I) or pharmaceutically acceptable salts are obtained after extraction and/or purification steps.

### DESCRIPTION OF FIGURES

FIG. 1: LDH assay by enzymatic reaction

FIG. 2: Cytotoxicity of calcein-AM (4 μM) and compound 1 in solution in Cremophor® EL [CAS 61791-12-6] (molar ratio 1/1) designated by the reference F4267 (40 μM and 1000 μM) on HCE-2 cells, expressed in % as a function of time after single contact (0=45 min, 24 h and 48 h).

FIG. 3: Images of HEL-299 cells obtained using the MVX10 macroscope under the FITC filter after staining: A: Calcein-AM (4 μM); B: F4267 (4 μM); C: F4267 (40 μM) (×2.5 and ×6.3 objectives for the first and second line respectively).

FIG. 4: Endothelial cell loss of human corneas preserved in organoculture and contacted with the product F4267 (40 μM) either repeatedly every day for 7 days or uniquely on DO, before each being exposed daily to the light under the FITC filter

FIG. 5: Viability measured in % by fluorescence analysis on human corneas, one of which is incubated daily with the product F4267 (images A and A′) and the other uniquely (images B and B′) on D0, but both exposed daily to light under the FITC filter. Images A and B represent the fluorescence after incubation with the product F4267 on D0 and images A′ and B′ represent the fluorescence after incubation with Calcein-AM at the end of the experiment on D7 to measure cell viability.

FIG. 6: Images of B4G12 cells obtained using an epifluorescence microscope under the FITC channel and after marking with calcein-AM (4 μM) and the product F4267 (40 μM) with and without rinsing with PBS according to example 5.

FIG. 7: Images of B4G12 cells obtained using an epifluorescence microscope under the FITC channel after marking for 5 days with calcein-AM (4 μM) and the product F4267 (40 μM), with and without rinsing with PBS, according to example 5.

FIG. 8: Images of B4G12 cells obtained using an epifluorescence microscope under the DAPI channel (405 nm) after marking for 5 days with calcein-AM (4 μM) and the product F4267 (40 μM), with and without rinsing with PBS, according to example 5.

### EXAMPLES

The examples given in the present application are illustrative and do not limit the scope of the invention.

### Example 1: Synthesis of Compounds

### 1. Abbreviations

AcOEt Ethyl acetate
DCE 1,2-dichloroethane
DCM Dichloromethane
DMAP 4-Dimethylaminopyridine
DMSO Dimethyl sulfoxide
PE Petroleum ether
HRMS High resolution mass spectrometry
NMR Nuclear magnetic resonance

### 2. Operating Procedures

The general synthesis of “fluorescein dicarbonate” compounds is carried out from fluorescein or corresponding derivatives. The synthesis is carried out in 2 steps: a) fluorescein or the corresponding derivative reacts with a reagent generating phosgene (phosgene, diphosgene or triphosgene) in the presence of base and the solvent is evaporated, b) the synthesis intermediate obtained reacts with an alcohol in the presence of a base to obtain the final compound of general formula (I).

### a) Synthesis Protocol in the Presence of Primary Alcohols

Diphosgene (6 eq.) then triethylamine (1 eq.) are successively added drop by drop to a suspension of fluorescein (1 eq.) in DCE. The reaction mixture is heated at 60° C. for 2 hours. The solvent is evaporated with a Schlenk line and the product is dried for several hours in the dark.

In another Schlenk tube, the primary alcohol (2 eq.) is dissolved in DCE and triethylamine (2 eq.) is added drop by drop. The fluorescein residue, previously obtained, is dissolved in DCE and the alcohol solution is added drop by drop over 10 minutes. The reaction mixture is heated for 3 hours at 70° C.

The reaction medium is poured into water and extracted with DCM. The organic extracts are combined, washed with water, then with a saturated aqueous NaCl solution, dried on MgSO₄, and the solvent is evaporated by rotary evaporation. The crude product is finally purified by column chromatography on silica gel (AcOEt/EP) to obtain the desired product.

### b) Synthesis Protocol in the Presence of Secondary Alcohols

Diphosgene (6 eq) then diisopropylethylamine (7 eq) are successively added drop by drop to a suspension of fluorescein (1 eq.) in DCE. The reaction mixture is heated at 60° C. for 2 hours. The solvent is evaporated with a Schlenk line and the product is dried for several hours in the dark.

In another Schlenk tube, the secondary alcohol (2 eq.) is dissolved in DCE. Diisopropylethylamine (2 eq.) and DMAP (1 eq.) are added. The fluorescein residue, previously obtained, is dissolved in DCE and the alcohol solution is added drop by drop over 10 minutes. The reaction mixture is heated for 3 hours at 70° C.

The reaction medium is poured into water and extracted with DCM. The organic extracts are combined, washed with water, then with a saturated aqueous NaCl solution, dried on MgSO₄, and the solvent is evaporated by rotary evaporation. The crude product is finally purified by column chromatography on silica gel (AcOEt/cyclohexane, then DCM/AcOEt) to obtain the desired product.

### 3. Synthesized Compounds

### Compound 1: Fluorescein Pentyl Dicarbonate

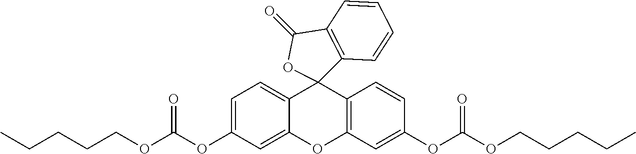 [SMILES: CCCCCOC(=O)Oc1ccc2c(c1)Oc1cc(OC(=O)OCCCCC)ccc1C21OC(=O)c2ccccc21]

Fluorescein pentyl dicarbonate is purified on silica gel (AcOEt/EP 15:80) and isolated in the form of a transparent oil (36%). ¹H NMR (500 MHz, CDCl₃) δ (ppm): 8.04 (dt, ³J=7.5 Hz, ⁴J=1.0 Hz, 1H), 7.69 (td, ³J=7.4 Hz, ⁴J=1.1 Hz, 1H), 7.64 (td, ³J=7.4 Hz, ⁴J=1.1 Hz, 1H), 7.22-7.14 (m, 3H), 6.91 (dd, ³J=8.7 Hz, ⁴J=2.3 Hz, 2H), 6.84 (d, ³J=8.7 Hz, 2H), 4.26 (t, ³J=6.7 Hz, 4H), 1.82-1.70 (m, 4H), 1.46-1.31 (m, 8H), 0.94 (t, ³J=6.7 Hz 6H). ¹³C NMR (126 MHz, CDCl₃) δ (ppm): 169.11, 153.08, 152.93, 152.44, 151.56, 135.34, 130.12, 129.07, 126.10, 125.31, 124.05, 117.26, 116.61, 109.93, 81.54, 69.41, 28.26, 27.81, 22.30, 13.95.

### Compound 2: Fluorescein Decyl Dicarbonate

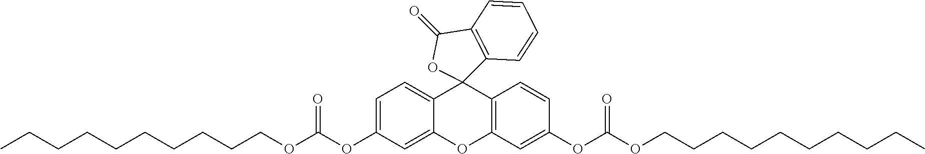 [SMILES: CCCCCCCCCCOC(=O)Oc1ccc2c(c1)Oc1cc(OC(=O)OCCCCCCCCCC)ccc1C21OC(=O)c2ccccc21]

Fluorescein decyl dicarbonate is purified on silica gel (AcOEt/EP 20:80) and isolated in the form of a transparent oil (77%). H NMR (500 MHz, DMSO-d₆) δ (ppm): 8.07 (dt, 3J=7.6 Hz, ⁴J=1.0 Hz, 1H), 7.83 (td, ³J=7.4 Hz, ⁴J=1.1 Hz, 1H), 7.77 (td, ³J=7.4 Hz, ⁴J=1.1 Hz, 1H), 7.46-7.36 (m, 3H), 7.06 (dd, ³J=8.7 Hz, ⁴J=2.4 Hz, 2H), 6.91 (d, ³J=8.7 Hz, 2H), 4.22 (t, ³J=6.6 Hz, 4H), 1.77-1.59 (m, 4H), 1.38-1.21 (m, 28H), 0.85 (t, ³J=6.6 Hz, 6H). ¹³C NMR (126 MHz, DMSO) δ (ppm): 168.85, 152.93, 152.60, 151.23, 136.52, 131.09, 129.75, 125.84, 125.55, 124.57, 118.52, 116.90, 110.48, 81.28, 69.39, 31.75, 29.38, 29.15, 29.04, 28.39, 25.59, 22.56, 14.42. HRMS (ESI-TOF) C₄₂H₅₂O₉ found: 701.3644 [M+H]⁺, calculated: 701.3684.

### Compound 3: Fluorescein Dicarbonate Diethyl (L-)Malate

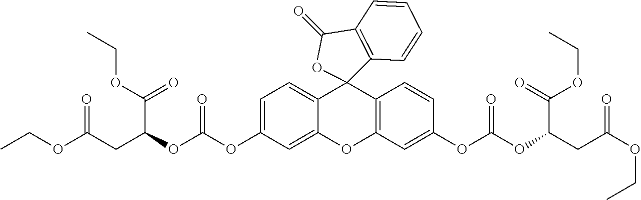 [SMILES: CCOC(=O)C[C@H](OC(=O)Oc1ccc2c(c1)Oc1cc(OC(=O)O[C@@H](CC(=O)OCC)C(=O)OCC)ccc1C21OC(=O)c2ccccc21)C(=O)OCC]

Fluorescein dicarbonate diethyl (L-) malate is purified on silica gel (AcOEt/cyclohexane 35:65, then DCM/AcOEt 95:5)¹H NMR (400 MHz, CDCl₃) δ (ppm): 8.06 (d, ³J=7.1 Hz, 1H), 7.78-7.60 (m, 2H), 7.27-7.12 (m, 3H), 6.97 (dd, ³J=8.7 Hz, ⁴J=2.4 Hz, 2H), 6.87 (d, ³J=8.8 Hz, 2H), 5.50 (t, ³J=6.1 Hz, 2H), 4.40-4.13 (m, 8H), 3.10-2.92 (m, 4H), 1.43-1.16 (m, 12H). ¹³C NMR (101 MHz, CDCl₃) δ (ppm): 169.02, 168.74, 168.08, 152.87, 152.18, 152.13, 151.48, 151.48, 135.37, 130.14, 129.12, 125.98, 125.32, 123.98, 117.09, 116.89, 109.82, 77.33, 77.02, 76.70, 72.27, 62.30, 61.43, 36.02, 14.13, 14.08.

### Compound 4: Fluorescein (L)-Malic Acid Dicarbonate

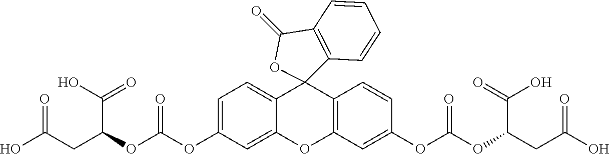 [SMILES: O=C(O)C[C@H](OC(=O)Oc1ccc2c(c1)Oc1cc(OC(=O)O[C@@H](CC(=O)O)C(=O)O)ccc1C21OC(=O)c2ccccc21)C(=O)O]

Fluorescein (L)-malic acid dicarbonate is purified on silica gel (AcOEt/EP 75:25, then DCM/AcOEt 97:3)¹H NMR (400 MHz, CDCl₃) δ (ppm): 8.08-8.01 (d, ³J=7.1 Hz 1H), 7.73-7.58 (m, 2H), 7.17 (d, ³J=7.1 Hz, 1H), 7.13 (d, ⁴J=2.2 Hz, 2H), 6.86 (dd, ³J=8.7 Hz, ⁴J=2.2 Hz, 2H), 6.81 (d, ³J=8.6 Hz, 2H), 4.36 (hept, ³J=6.7 Hz, 2H), 3.35 (d, ³J=7.4 Hz, 4H).

### Example 2: In Vitro Study on Adherent Cells of the “Research Laboratory Test” Type

### 1. Material and Methods

### Cytotoxicity

The cytotoxicity of a solution of compound 1 in Cremophor® EL [CAS 61791-12-6](molar ratio 1/1), designated under the reference F4267, compared to that of calcein-AM, was evaluated on the human corneal epithelium cell line immortalized by SV40, HCE-2 (ATCC CRL-11135) using two solutions of F4267 of concentrations [40 μM] and [1000 μM] or calcein-AM (FP-F19820, Interchim) [4 μM] diluted in optiMEM (11058021, Gibco, ThermoFisher) and deposited for 45 minutes on the cells (100 μL per well seeded 24 hours previously at 1.10⁵ cells/mL [3788, Corning, surface: 0.32 cm²]. After 45 minutes of contact with compound 1 or with calcein-AM, the cells were rinsed with their culture medium, then returned to culture in an oven with 5% CO₂, at 37° C. Cytotoxicity was measured by dosing lactate dehydrogenases (LDH) released into the supernatant using the CyQUANT LDH Cytotoxicity Assay kit (C20301, ThermoFisher, see cytotoxicity test below) and expressed as % of dead cells using an internal standard. Cytotoxicity was evaluated 45 minutes (before rinsing), 24 h and 48 h after the start of incubation (3 wells per condition, n=1 experiments).

### Cytotoxicity Test

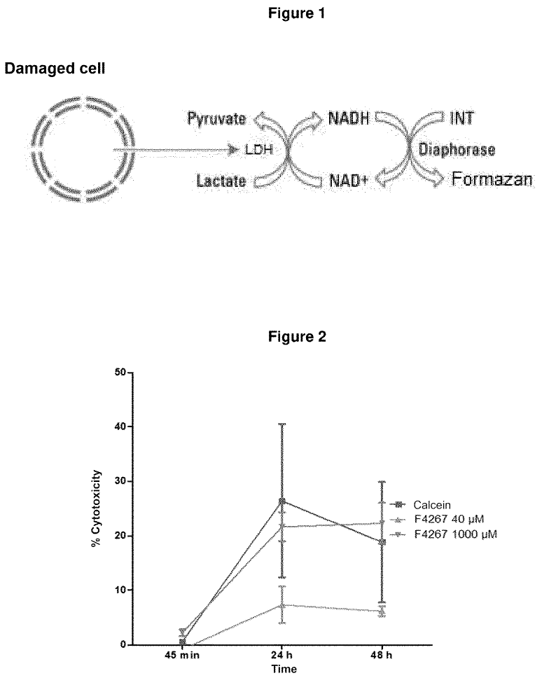

Lactate DeHydrogenases (LDH) are a family of ubiquitous cytosolic enzymes present in almost all cell types. Membrane damage causes a release of LDH into the external environment. There is a direct correlation between cellular cytotoxicity which causes membrane damage and the amount of LDH present in the extracellular environment. In the cytotoxicity test, LDH is measured by the enzymatic reaction shown in FIG. 1. LDH transforms pyruvate into lactate at the same time as there is a reduction of NAD+ into NADH. The NADH thus produced reacts with fluorescent or luminescent molecules.

The cytotoxicity test was carried out with tetrazolium salt (INT) which is converted into formazan. The staining was then studied using a spectrophotometer. The concentration of formazan being proportional to the amount of LDH present in the medium, this was quantified by an absorbance reading at 555 nm, against a blank measurement at 650 nm to eliminate the parasitic absorbance of the medium. The percentage of dead cells is then calculated from the formula below, with the maximum LDH activity being that of a well from which all cells have been lysed, and the basal LDH activity, that of a non-treated well.

$$\% dead cells = ( \frac{L DH activity of the treated well - basal L DH activity}{maximum L DH activity - basal L DH activity} ) * 100$$

### Quality of Marking

The quality of cytoplasmic marking by compound 1 on cells was evaluated on the adherent line of normal human fetal lung fibroblasts HEL299 [ATCC CCL-137], because they form a cellular monolayer (a single thickness facilitating the evaluation of cellular morphology and enumeration). Cells were seeded at 1.10⁵ cells/mL 72 h before marking, then marked with the solution of compound 1 designated by the reference F4267 at [4 μM] and [40 μM] or with a solution of Calcein-AM [4 μM], then observed in a sterile Petri dish under the FITC filter of a macroscope (macro-zoom microscope, MVX10, Olympus, Tokyo, Japan) (2 wells per condition, n=1 experiments).

### 2. Results

### Cytotoxicity

At 45 min, the cytotoxicity (%) induced on HCE-2 was very low: 0.6±0.5% for calcein-AM, 0.8±0.5% for F4267 at 40 μM and 2.2±0.6% for F4267 1000 μM.

At 24 h, the cytotoxicities were 26.4±14.1% for calcein-AM, 7.3±3.4% for F4267 at 40 μM and 21.6±2.7% for F4267 at 1000 μM.

At 48 h, the cytotoxicities were 18.8±11.1% for calcein-AM, 6.1±0.9% for F4267 at 40 μM and 22.3±3.8% for F4267 at 1000 μM (FIG. 2).

### Quality of Marking

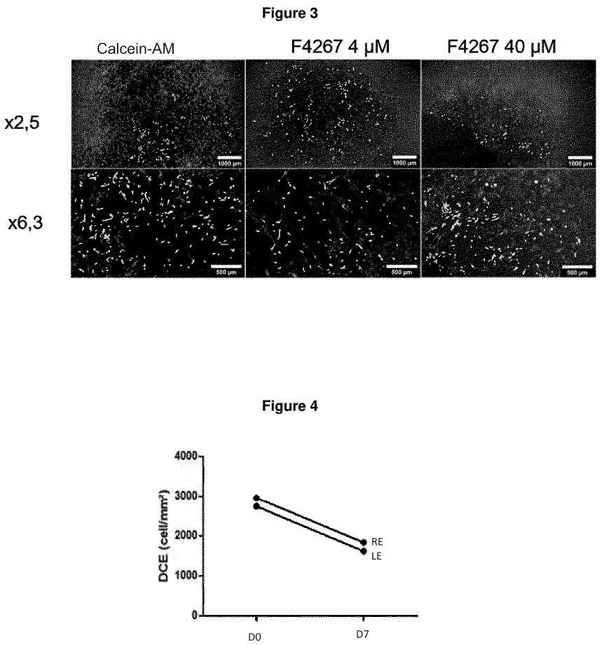

The marking of HEL299 cells allowed to visualize the cell cytoplasms in a similar way with the Calcein-AM solution [4 μM] and with the F4267 solution at [4 μM] and [40 μM] respectively (FIG. 3).

### Example 3: Ex Vivo Study on Endothelium of Whole Corneas, of “Cornea Bank Test” Type

### 1. Material and Methods

### Cytotoxicity

The cytotoxicity of compound 1 (solution F4267) was evaluated on the endothelium of whole corneas, using pairs of human corneas, preserved in organoculture and rejected for transplantation by a cornea bank. The 2 corneas from the same donor have the same biological features and constitute the best comparator of each other. Endothelial Cell Density (ECD) was first evaluated before experimentation using cell counting by image analysis [Cell Tissue Bank, 2017, 18 (2), 185-191, Jumelle and al.], then the endothelium of both corneas was contacted with a solution of F4267 [40 μM] for 45 minutes at room temperature, under sterile conditions [Invest Ophthalmol Vis Sci. 2011, 52 (8), 6018-6025, Pipparelli and al.]. The corneas were then observed using the MVX10 macroscope with the ×0.8 objective under the FITC filter, then returned to organoculture. In order to evaluate the toxicity by repeated incubation, the cornea of the right eye (RE) was contacted daily with a solution of F4267 [40 μM in OptiMEM, 45 minutes, room temperature] for 7 days while the cornea of the left eye (LE) was only incubated with optiMEM (solvent alone). The corneas were exposed daily to blue light from the FITC filter (same intensity of XCite lamp at 12/100) under the ×0.8 objective of the MVX10 macroscope. The DCE was measured again on D7 using the Hoechst-Ethidium-Calcein-AM staining (Invest Ophthalmol Vis Sci. 2011, 52 (8), 6018-6025, Pipparelli and al) (1 pair of corneas, n=1).

### Quality of Marking

The quality of cytoplasmic marking by compound 1 (solution F4267) on corneal endothelium was compared to that of Calcein-AM by staining paired corneas. The endothelial surface of the corneas was incubated for 45 minutes either with the F4267 solution (40 μM on the left eye, LE) or with Calcein-AM (4 μM on the right eye, RE) following a validated staining protocol. (Invest Ophthalmol Vis Sci. 2011, 52 (8), 6018-6025, Pipparelli and al.) (n=2 pairs of corneas).

### 2. Results

### Cytotoxicity

The DCE in viable cells on D7, after daily contact of the cornea of the RE with the product F4267 followed by observation at low magnification for 7 days, was 1832 cells/mm². This DCE was 1612 cells/mm² for a single contact of the cornea of the LE but also with daily exposure to light. Cell loss is therefore comparable, indicating that repeated incubation is well tolerated. (FIG. 4).

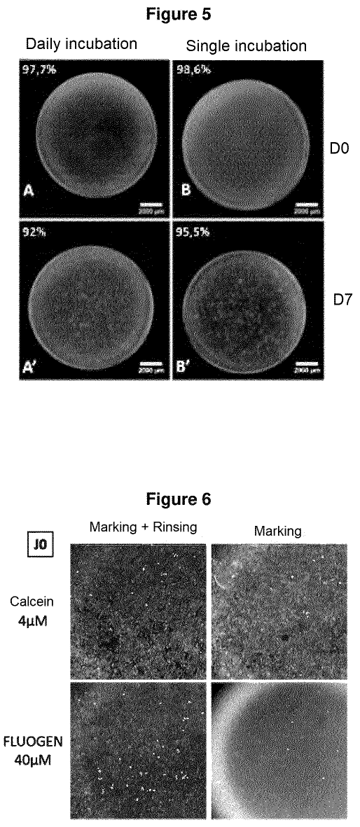

The images in FIG. 5 illustrate the viability measured in % after marking with Calcein-AM on D7 by fluorescence image analysis: cornea A (RE) repeatedly contacted every day for 7 days with the product F4267, cornea B (LE) contacted only on DO but both exposed daily to light under the FITC filter.

### Quality of Marking

The product F4267 [40 μM] allows a marking differentiating dead cells from living cells (FIGS. 5A and 5B, on DO). In addition, it has been shown that the product F4267 [40 μM] allows a marking differentiating dead cells from living cells in a manner similar to Calcein-AM [4 μM] on the entire cornea.

### Example 4: Marking Different Cell Types

Corneal endothelial cells (B4G12), fibroblasts (Hel-299), and corneal epithelial cells (HCE-2) were cultured on an 8-well culture slide (LabTek, 177445) for 5 days in corresponding culture media described below.

B4G12 culture medium: Gentamycin, OptiMEM, SVFD, CaICl₂, Ascorbic acid, EGF, Chondroitin sulfate, SB203580, Y27632.
HCE-2 culture medium: DMEM GlutaMax, F12-HAM, SVFD, Antibiotic/mycotic, EGF.
Hel-299 culture medium: DMEM GlutaMax, SVFD, ATB/M

Upon confluence, cell marking is carried out for 45 minutes with solutions comprising Calcein-AM or the product F4267 at different concentrations (solutions A, B, C and D), before rinsing with PBS. Then the culture wells are removed, and the slide is mounted with a coverslip and Vectashield.

Solution A: calcein-AM (4 μM), Hoescht ( 1/200), ethydium ( 1/500)
Solution B: F4267 (40 μM), Hoescht ( 1/200), ethydium ( 1/500)
Solution C: F4267 (4 μM), Hoescht ( 1/200), ethydium ( 1/500)
Solution D: F4267 (400 μM), Hoescht ( 1/200), ethydium ( 1/500)

The slides are then observed under a macroscope with ×0.8 and ×6.3 magnification and with an epifluorescence microscope with ×10 and ×60 magnification. The different wells are observed under the same conditions at each magnification to allow a comparison of the intensities and the quality of the marking with identical image processing.

The cytoplasms of living cells, regardless of the cell type, are clearly visible with compound F4267.

### Example 5: Evaluation of the Impact of Cell Marking on Cell Viability

The experiment is carried out with HCEC-B4G12 corneal endothelial cells (CVCL_2065). The cells are thawed and cultured in their culture medium. The cells are trypsinized once a week before being seeded in wells of 24-well slides at a concentration of 1.10⁵ cells/mL. The cells are then incubated for 5 days at 37° C., 5% CO₂ until 90% confluence.

The different wells are stained daily with Calcein-AM (4 μM) or product F4267 (40 μM), according to the following conditions: marking for 45 minutes, then rinsing with PBS or marking for 45 minutes without rinsing. The marking and possibly rinsing step is repeated every day for 4 successive days, then after 2 days without marking, the marking is repeated again for 2 consecutive days.

The marking steps are carried out in duplicate. Afterwards, each marking, a well for each of the conditions is observed by epifluorescence microscopy (Olympus IX81 Microscope, FITC channel, ×4 objective, L: 25, Texp: 142.7 ms), while the second well (control) is kept in the darkness in order to evaluate the impact of fluorescence observation at the end of the study. On the last day of the study, the marking is carried out with calecin-AM (4 μM) in the presence of Hoescht ( 1/500) or the product F4267 (40 μM) in the presence of Hoescht ( 1/500), and the wells are imaged in MIA×4 (reconstruction of several fields to obtain a complete image of the well).

The marking steps are carried out in a clean room under PSM to ensure sterility, and the culture dishes are parafilmed during observation to limit possible contamination.

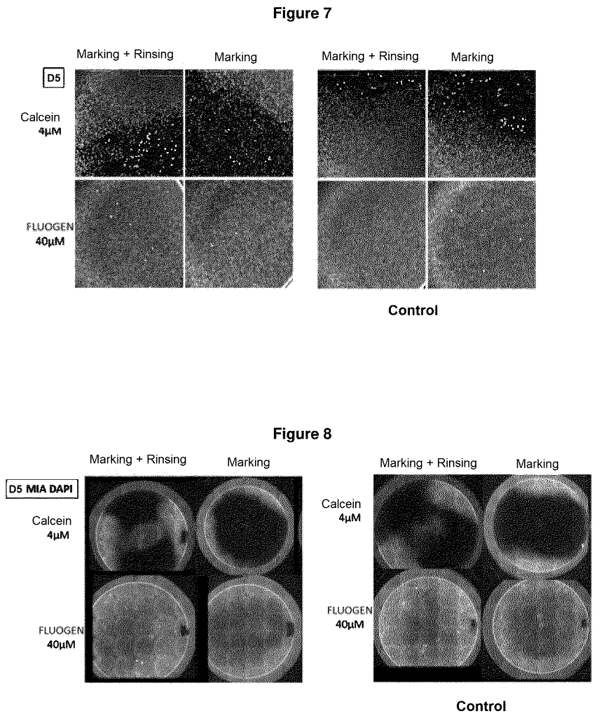

FIGS. 6 and 7 represent the cell layers observed on DO, therefore the first day of marking, and on D5, that is to say the last day of marking. It appears that the cell layers of cells marked with Calcein-AM are less dense whether for the experiments in the presence or absence of rinsing, unlike the cell layers whose cells were marked with F4267.

FIG. 8 corresponding to the images of the cells under the DAPI channel confirms the absence of cell layer for the cells marked with calcein-AM.

These different observations conclude that the product F4267 appears to be non-toxic, or at least less toxic than calcein-AM for cells, since no negative influence of the marker F4267 on the cell layer is demonstrated.

1. A method of cellular marking of cells comprising the steps of contracting the cells with a compound of general formula (I), 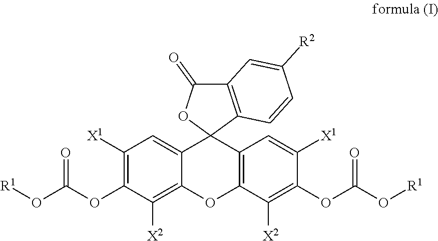
wherein:
R¹ is selected from
a C₃-C₂₀ alkyl,
a polyethylene glycol or a polypropylene glycol,
malic acid derivatives and corresponding esters,
sugars, and
natural polysaccharides;
X¹ is selected from H, Cl, F, Br and NO₂;
X² is selected from H, Cl, Br and N(CH₂CO₂H)₂;
R² is selected from H, NCS and CO₂R³; and
R³ is selected from H or a C₁-C₃ alkyl,
and its pharmaceutically acceptable salts,
assessing the presence of stain or fluorescence in said cells.

2. The method according to claim 1, wherein R¹ is a C₅-C₁₀ alkyl.

3. The method according to claim 1, wherein R² is hydrogen.

4. The method according to claim 1, wherein X² is hydrogen.

5. The method according to claim 1, wherein X² is hydrogen.

6. The method according to claim 1, wherein the cell marking is carried out in vitro.

7. The method according to claim 1, wherein the cell marking is carried out in vivo or ex vivo.

8. The method according to claim 1 to 7, wherein the cells are endothelial cells.

9. The method according to claim 8, wherein the cells are corneal endothelial cells.

10. A pharmaceutical composition comprising a compound of formula (I) defined in claim 1 and an excipient and/or a pharmaceutically acceptable carrier.

11. The pharmaceutical composition according to claim 10, wherein the composition is an aqueous composition.

12. The pharmaceutical composition according to claim 10, wherein the composition is in the form of a gel or a film.

13. The pharmaceutical composition according to claim 11, wherein the excipient and/or the carrier is suitable for ophthalmic use.

14. A method for preparing a compound of formula (I) 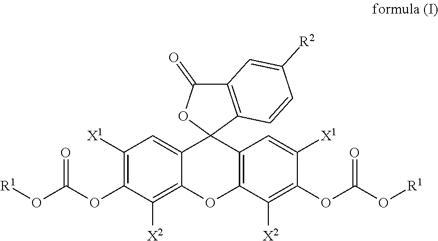
wherein
R¹ is selected from
a C₃-C₂₀ alkyl,
a polyethylene glycol or a polypropylene glycol,
malic acid derivatives and corresponding esters,
sugars, and
natural polysaccharides;
X¹ is selected from H, Cl, F, Br and NO₂;
X² is selected from H, Cl, Br and N(CH₂CO₂H)₂;
R² is selected from H, NCS and CO₂R³; and
R³ is selected from H or a C₁-C₃ alkyl
and its pharmaceutically acceptable salts,
wherein the method comprises the steps of:
a) Reaction between a diphenol (II), 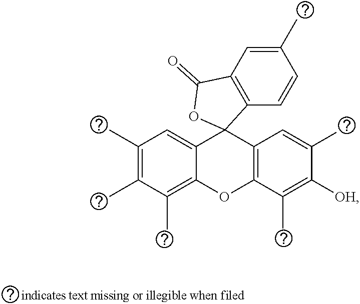
with phosgene or a derivative thereof in the presence of base leading to the formation of the synthesis intermediate of formula (III) 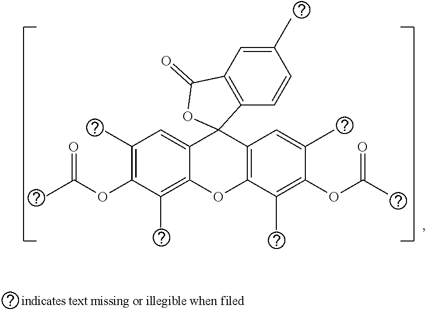
Reaction of the intermediate (III), 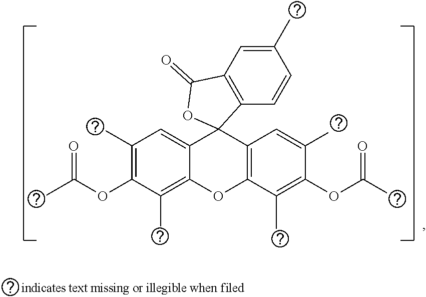
with an alcohol of formula (IV), R¹OH, in the presence of base to obtain the compound of formula (I).

15. A method for assessing the cellular viability of cells which comprises the steps of contacting the cells with a compound of general formula (I) 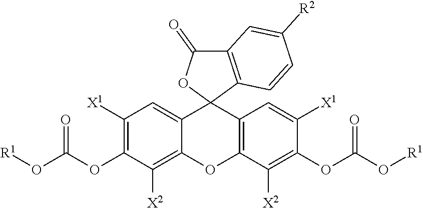
wherein
R¹ is selected from
a C₃-C₂₀ alkyl,
a polyethylene glycol or a polypropylene glycol,
malic acid derivatives and corresponding esters,
sugars, and
natural polysaccharides;
X¹ is selected from H, Cl, F, Br and NO₂;
X² is selected from H, Cl, Br and N(CH₂CO₂H)₂;
R² is selected from H, NCS and CO₂R³; and
R³ is selected from H or a C₁-C₃ alkyl
and its pharmaceutically acceptable salts,
assessing the presence of staining or fluorescence of the cells, the staining or fluorescence being indicative of the presence of living cells.

16. The method according to claim 15, wherein R¹ is a C₅-C₁₀ alkyl, and/or R² is hydrogen and/or X¹ is hydrogen and/or X² is hydrogen.

17. The method according to claim 15, wherein the cell marking is carried out in vitro.

18. The method according to claim 15, wherein the cell marking is carried out in vivo or ex vivo.

19. The method according to claim 15, wherein the cells are endothelial cells.

20. The method according to claim 15, wherein the cells are corneal endothelial cells.
